(19) United States
(12) Patent Application Publication
(10) Pub. No.: US 2024/0304289 A1
(43) Pub. Date: Sep. 12, 2024

(54) CUSTOMIZED NEUROSTIMULATION TREATMENT INFORMATION BASED ON USER INTERACTIONS

(71) Applicant: Boston Scientific Neuromodulation Corporation, Valencia, CA (US)

(72) Inventors: Lisa Denise Moore, Glendal, CA (US); Bradley Lawrence Hershey, Carrollton, TX (US)

(21) Appl. No.: 18/590,679

(22) Filed: Feb. 28, 2024

Related U.S. Application Data
(60) Provisional application No. 63/451,008, filed on Mar. 9, 2023.

(51) Int. Cl. G16H 10/20; A61N 1/36; G16H 40/40

(52) U.S. Cl. CPC G16H 10/20; A61N 1/36132; A61N 1/36139; G16H 40/40

(57) ABSTRACT

A system (e.g., computing system) for analyzing user interaction associated with a neurostimulation treatment may include functionality to: receive user interaction data associated with a patient undergoing the neurostimulation treatment, with the user interaction data indicating attributes of one or more user interactions in a software platform (e.g., patient computing device) that collects user feedback relating to the neurostimulation treatment; identify attributes of the user interactions from the user interaction data; generate a task to collect additional user input relating to the neurostimulation treatment, with the task being customized to the patient based on the attributes of the user interactions; and control an interaction workflow to perform the task in the software platform. Further functionality may: modify the interaction workflow for the neurostimulation treatment; identify a patient state resulting from the neurostimulation treatment; or cause a change in a closed-loop programming therapy for the neurostimulation treatment.

### CLAIM OF PRIORITY

This application claims the benefit of U.S. Provisional Application No. 63/451,008 filed on Mar. 9, 2023, which is hereby incorporated by reference in its entirety.

### TECHNICAL FIELD

This document relates generally to data processing obtained in connection with the use of medical devices, and more particularly, to systems, devices, and methods for customizing information collection and processing information in connection with patient uses of implanted electrical stimulation, including the collection and processing of user-provided interactions associated with neurostimulation treatments for pain, movement disorders, and/or management of such conditions.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). A neurostimulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system that uses similar pulses of electricity at particular locations in the brain to reduce symptoms of essential tremors, Parkinson's disease, psychological disorders, or the like.

Various approaches are being developed to enable personalized programming and optimized forms of programming used by neurostimulation systems, including partially or fully automated forms of generating or delivering specific neurostimulation parameters known as closed-loop programming. Some closed-loop programming approaches can, for example, customize changes to stimulator programs, such as to suggest or automatically select a program when a particular condition is identified. However, personalized and closed-loop programming typically requires a robust and extensive set of data inputs, to accurately learn characteristics about the individual patient and to adapt to the user's particular behavior. Although there has been a progressive increase in digital connectivity and approaches to track user behavior and collect user data, including from patent questionnaires and customized questions, it is often difficult to collect useful information from a particular patient or a population of patients on a consistent basis. For example, while some patients may be willing to provide feedback every day in an online questionnaire, other patients may decide to provide feedback only a few times a week (or less), and may want to minimize the amount of interaction and feedback data that is provided.

### SUMMARY

An example (e.g., “Example 1”) of a system (e.g., a computing system or other programmed device) may be configured to analyze user interaction associated with a neurostimulation treatment provided by a neurostimulation device, with such system including: one or more processors; and one or more memory devices including instructions, which when executed by the one or more processors, cause the one or more processors to: receive user interaction data associated with a patient undergoing the neurostimulation treatment, wherein the user interaction data indicates attributes of one or more user interactions in a software platform that collect user feedback relating to the neurostimulation treatment; identify attributes of the one or more user interactions, from the user interaction data; generate a task to collect additional user input relating to the neurostimulation treatment, the task being customized to the patient based on the attributes of the one or more user interactions; and control an interaction workflow to perform the task in the software platform.

In Example 2, the subject matter of Example 1 optionally includes subject matter where the user feedback is collected from the patient using one or more questions during each session of the one or more user interactions, and wherein one or more answers corresponding to the one or more questions provide a state of the patient undergoing the neurostimulation treatment.

In Example 3, the subject matter of Example 2 optionally includes subject matter where the attributes of the one or more user interactions relate to at least one of: a number of tasks completed in each session; a number of questions completed in each session; an ongoing or historical usage of the software platform; an ongoing or historical pattern of use of the software platform; an amount of time of engagement in each session; an amount of time of engagement with respective types of questions in each session; a time of day of engagement in each session; or a pattern of engagement in each session.

In Example 4, the subject matter of any one or more of Examples 1-3 optionally includes subject matter where the task includes multiple questions to provide to the patient in a subsequent user interaction, wherein the interaction workflow controls presentation of the multiple questions to the patient, and wherein a number, type, or order of the multiple questions is customized to the patient based on the attributes.

In Example 5, the subject matter of Example 4 optionally includes subject matter where a priority is determined for each question of the multiple questions, and wherein the number, type, or order of the multiple questions is further customized to the patient based on the priority of each question.

In Example 6, the subject matter of any one or more of Examples 4-5 optionally includes subject matter where a complexity is determined for each question of the multiple questions, and wherein the number, type, or order of the multiple questions is further customized to the patient based on the complexity of each question.

In Example 7, the subject matter of any one or more of Examples 1-6 optionally includes subject matter where the task includes a scheduled time period to be presented in the software platform, wherein the scheduled time period includes a start time and an end time, and wherein the scheduled time period is customized to the patient based on the attributes.

In Example 8, the subject matter of any one or more of Examples 1-7 optionally includes subject matter where the interaction workflow is a patient interaction workflow to occur with the patient, and wherein the software platform is implemented on a patient computing device provided by a personal computer, tablet, smartphone, remote control, or wearable device.

In Example 9, the subject matter of any one or more of Examples 1-8 optionally includes subject matter where the interaction workflow is a caregiver workflow to occur with a caregiver associated with the patient or a clinician workflow to occur with a clinician associated with the patient, and wherein the instructions further cause the one or more processors to cause the interaction workflow to collect data from the caregiver or the clinician relating to the patient.

In Example 10, the subject matter of any one or more of Examples 1-9 optionally includes subject matter where the instructions further cause the one or more processors to: determine a variability between the attributes of the one or more user interactions and expected attributes of the one or more user interactions, wherein the task is customized to the patient based on the determined variability.

In Example 11, the subject matter of any one or more of Examples 1-10 optionally includes subject matter where the instructions further cause the one or more processors to:

identify a patient state based on one or more of the user feedback or objective data associated with the patient; and associate the patient state with at least one neurostimulation program used for the neurostimulation treatment; wherein the interaction workflow is modified based on the patient state or the at least one neurostimulation program.

In Example 12, the subject matter of Example 11 optionally includes subject matter where the instructions further cause the one or more processors to: control a closed-loop programming therapy of the neurostimulation device based on the identified patient state, wherein the patient state relates to one or more of: sleep, actigraphy, accelerometry, pain, movement, stress, disease-related symptoms, emotional state, medication state, or activity during use of the at least one neurostimulation program.

In Example 13, the subject matter of Example 12 optionally includes subject matter where the closed-loop programming therapy causes an automatic change to neurostimulation programming settings on the neurostimulation device, and wherein the automatic change to the neurostimulation programming settings controls one or more of: pulse patterns, pulse shapes, a spatial location of pulses, electric fields or activating functions of active electrodes, waveform shapes, or a spatial location of waveform shapes, for modulated energy provided with a plurality of leads of the neurostimulation device.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a computing device to analyze user interaction associated with a neurostimulation treatment, the device comprising: one or more processors; and one or more memory devices comprising instructions, which when executed by the one or more processors, cause the one or more processors to: receive user interaction data associated with a patient undergoing the neurostimulation treatment, wherein the user interaction data indicates attributes of one or more user interactions in a software platform that collect user feedback relating to the neurostimulation treatment; identify attributes of the one or more user interactions, from the user interaction data; generate a task to collect additional user input relating to the neurostimulation treatment, the task being customized to the patient based on the attributes of the one or more user interactions; and control an interaction workflow to perform the task in the software platform.

In Example 17, the subject matter of Example 16 optionally includes subject matter where the user feedback is collected from the patient using one or more questions during each session of the one or more user interactions, and wherein one or more answers corresponding to the one or more questions provide a state of the patient undergoing the neurostimulation treatment.

In Example 18, the subject matter of Example 17 optionally includes subject matter where the attributes of the one or more user interactions relate to at least one of: a number of tasks completed in each session; a number of questions completed in each session;

an ongoing or historical usage of the software platform; an ongoing or historical pattern of use of the software platform; an amount of time of engagement in each session; an amount of time of engagement with respective types of questions in each session; a time of day of engagement in each session; or a pattern of engagement in each session.

In Example 19, the subject matter of any one or more of Examples 16-18 optionally includes subject matter where the task includes multiple questions to provide to the patient in a subsequent user interaction, wherein the interaction workflow controls presentation of the multiple questions to the patient, and wherein a number, type, or order of the multiple questions is customized to the patient based on the attributes.

In Example 20, the subject matter of Example 19 optionally includes subject matter where a priority is determined for each question of the multiple questions, and wherein the number, type, or order of the multiple questions is further customized to the patient based on the priority of each question.

In Example 21, the subject matter of any one or more of Examples 19-20 optionally includes subject matter where a complexity is determined for each question of the multiple questions, and wherein the number, type, or order of the multiple questions is further customized to the patient based on the complexity of each question.

In Example 22, the subject matter of any one or more of Examples 16-21 optionally includes subject matter where the task includes a scheduled time period to be presented in the software platform, wherein the scheduled time period includes a start time and an end time, and wherein the scheduled time period is customized to the patient based on the attributes.

In Example 23, the subject matter of any one or more of Examples 16-22 optionally includes subject matter where the interaction workflow is a patient interaction workflow to occur with the patient, and wherein the software platform is implemented on a patient computing device provided by a personal computer, tablet, smartphone, remote control, or wearable device.

In Example 24, the subject matter of any one or more of Examples 16-23 optionally includes subject matter where the instructions further cause the one or more processors to: identify a patient state based on one or more of the user feedback or objective data associated with the patient; cause a change to the interaction workflow based on the identified patient state; and cause a change in a closed-loop programming therapy of a neurostimulation device based on the identified patient state, wherein the patient state relates to one or more of: sleep, actigraphy, accelerometry, pain, movement, stress, disease-related symptoms, emotional state, medication state, or activity during use of at least one neurostimulation program.

In Example 25, the subject matter of Example 24 optionally includes subject matter where the closed-loop programming therapy causes an automatic change to neurostimulation programming settings on the neurostimulation device, and wherein the automatic change to the neurostimulation programming settings controls one or more of: pulse patterns, pulse shapes, a spatial location of pulses, electric fields or activating functions of active electrodes, waveform shapes, or a spatial location of waveform shapes, for modulated energy provided with a plurality of leads of the neurostimulation device.

Example 26 is a method for analyzing user interaction associated with a neurostimulation treatment, comprising: receiving user interaction data associated with a patient undergoing the neurostimulation treatment, wherein the user interaction data indicates attributes of one or more user interactions in a software platform that collect user feedback relating to the neurostimulation treatment; identifying attributes of the one or more user interactions, from the user interaction data; generating a task to collect additional user input relating to the neurostimulation treatment, the task being customized to the patient based on the attributes of the one or more user interactions; and controlling an interaction workflow to perform the task in the software platform.

In Example 27, the subject matter of Example 26 optionally includes subject matter where the user feedback is collected from the patient using one or more questions during each session of the one or more user interactions, and wherein one or more answers corresponding to the one or more questions provide a state of the patient undergoing the neurostimulation treatment.

In Example 28, the subject matter of Example 27 optionally includes subject matter where the attributes of the one or more user interactions relate to at least one of: a number of tasks completed in each session; a number of questions completed in each session;

an ongoing or historical usage of the software platform; an ongoing or historical pattern of use of the software platform; an amount of time of engagement in each session; an amount of time of engagement with respective types of questions in each session; a time of day of engagement in each session; or a pattern of engagement in each session.

In Example 29, the subject matter of any one or more of Examples 26-28 optionally includes subject matter where the task includes multiple questions to provide to the patient in a subsequent user interaction, wherein the interaction workflow controls presentation of the multiple questions to the patient, and wherein a number, type, or order of the multiple questions is customized to the patient based on the attributes.

In Example 30, the subject matter of Example 29 optionally includes subject matter where a priority is determined for each question of the multiple questions, and wherein the number, type, or order of the multiple questions is further customized to the patient based on the priority of each question.

In Example 31, the subject matter of any one or more of Examples 29-30 optionally includes subject matter where a complexity is determined for each question of the multiple questions, and wherein the number, type, or order of the multiple questions is further customized to the patient based on the complexity of each question.

In Example 32, the subject matter of any one or more of Examples 26-31 optionally includes subject matter where the task includes a scheduled time period to be presented in the software platform, wherein the scheduled time period includes a start time and an end time, and wherein the scheduled time period is customized to the patient based on the attributes.

In Example 33, the subject matter of any one or more of Examples 26-32 optionally includes subject matter where the interaction workflow is a patient interaction workflow to occur with the patient, and wherein the software platform is implemented on a patient computing device provided by a personal computer, tablet, smartphone, remote control, or wearable device.

In Example 34, the subject matter of any one or more of Examples 26-33 optionally include identifying a patient state based on one or more of the user feedback or objective data associated with the patient; causing a change to the interaction workflow based on the identified patient state; and causing a change in a closed-loop programming therapy of a neurostimulation device based on the identified patient state, wherein the patient state relates to one or more of: sleep, actigraphy, accelerometry, pain, movement, stress, disease-related symptoms, emotional state, medication state, or activity during use of at least one neurostimulation program.

In Example 35, the subject matter of Example 34 optionally includes subject matter where the closed-loop programming therapy causes an automatic change to neurostimulation programming settings on the neurostimulation device, and wherein the automatic change to the neurostimulation programming settings controls one or more of: pulse patterns, pulse shapes, a spatial location of pulses, electric fields or activating functions of active electrodes, waveform shapes, or a spatial location of waveform shapes, for modulated energy provided with a plurality of leads of the neurostimulation device.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a data processing system to collect and analyze healthcare-related data in connection with the use of neurostimulation programs and closed-loop neurostimulation programming.

FIG. 2 illustrates a neuromodulation system as an example of a medical device system, according to an example.

FIG. 3 further illustrates a neuromodulation system as an ambulatory medical device, such as implemented by a neurostimulation treatment, according to an example.

FIG. 4 illustrates a neuromodulation device connected to a programming device, according to an example.

FIG. 5 illustrates data interactions with a data processing system for operation of a neuromodulation device with user interaction and task-based workflows, according to an example.

FIG. 6 illustrates data types and processing logic associated with data input and output for user interaction data processing, according to an example.

FIG. 7 illustrates a workflow for the generation, delivery, and evaluation of user interaction events, according to an example.

FIGS. 8A and 8B illustrate example data properties associated with a user interaction task and user interaction event, according to an example.

FIGS. 9A and 9B illustrate user interfaces with input and output functionalities, according to an example.

FIG. 10 illustrates a data processing flow for controlling the neurostimulation treatment of a human patient, based on the user interaction data processing operations, according to an example.

FIG. 11 illustrates a flowchart of a processing method to analyze user interaction activities in connection with a neurostimulation treatment, according to an example.

FIG. 12 illustrates a block diagram of a system (e.g., a computing system) for performing analysis of user interaction data, according to an example.

FIG. 13 illustrates a block diagram of an embodiment of a system (e.g., a computing system) implementing neurostimulation programming circuitry to cause programming of an implantable electrical neurostimulation device, according to an example.

FIG. 14 is a block diagram illustrating a machine in the example form of a computer system, within which a set or sequence of instructions may be executed to cause the machine to perform any one of the methodologies discussed herein, according to an example.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments of the present subject matter relate to user interfaces, data communications, data processing operations, customized workflows, and computing systems and devices used in connection with a neurostimulation programming system, neurostimulation devices, and associated data processing systems. As an example, aspects refer to user interface screens and software-initiated processes that attempt to assess a user's behavior in a particular software application (“app”) that coordinates user interaction tasks such as assigned questionnaires, surveys, etc. The user interaction data, once collected, can be used to identify personalized approaches that assign certain tasks, ask a particular type or number of questions, and generally collect information from a particular user (i.e., a patient undergoing neurostimulation or their caregiver). The user interaction data can be used to change or alter the presentation of tasks and information, to increase the likelihood of obtaining more effective user responses. Ultimately, the coordination of interaction tasks and the collection of information from a patient can provide valuable feedback information that can effect a neurostimulation treatment workflow, including to drive programming recommendations, reprogramming changes, alerts or notifications, or a variety of other operations associated with neurostimulation programming.

Digital tools that request patient feedback are most effective when they achieve high levels of repeatable patient interaction. Often, user interaction that is observed by most existing approaches will ask a patient to voluntarily provide as much information as they are willing to share. While some patients may log into a software platform every day and answer questions for a long period of time (e.g., 30 minutes or more), other patients may only log into the software platform a few times a week and may want to devote as little as possible time (e.g., only a few minutes) to feedback. The following includes systems and methods to analyze usage and user interaction data, to determine a patient's likely user interaction activity patterns and preferences, which then can be used to generate customized tasks and question workflows. The following also includes approaches for measuring user interactions and altering the presentation of application-associated tasks or other dynamic items based on the user's current and prior interaction activities. The resulting presentation of materials in a software app, website, or platform can then prioritize the collection of critical data, while attempting to maximize user engagement and retention. This can have the accompanying benefits of reducing user interaction burden, improving the content and types of data presentation, capturing more accurate and more useful data, and ultimately using such data to improve the outcomes of neurostimulation programming and treatment.

In an example, a user interface is provided in a smartphone or tablet app, website, or another dynamic interface that records user interaction data. As used herein, such “user interaction” data includes user-initiated or user-controlled entry of data via text, graphical, voice, audiovisual, or other input formats. User interaction data also includes metadata and background data related to the amount of usage of a user interface, the types of actions taken or the time spent on a software app or respective user interface screens, time spent on different types of tasks, the amount or characteristics of different types of user input, or the like.

Various forms of data processing may occur based on the collected user interaction data, including data processing that leads to new or changed question or interaction workflows, recommendations and suggestions, alerts or warnings, and data-driven activities that can lead to new reprogramming or device operation workflows. As one example, user interaction data may be used to generate a targeted sequence of questions about a particular patient's treatment, to maximize the amount of relevant data collected from the particular patient. Also, such user interaction data can be used to improve or enhance a closed-loop programming system, to automatically select or recommend therapies from new programming of a neurostimulation device based on the user interaction answers that are provided. Accordingly, the presently disclosed data processing can lead to a variety of optimizations in the use and deployment of new programs, modification of existing programs, optimization of program usage and schedules, and other neurostimulation device improvements.

The following describes a “data collection platform”, “data processing system”, and “data service” that generally refers to portions of a compute platform (e.g., a combination of hardware, firmware and software) with a set of capabilities for collecting, processing, and generating user interaction data in connection with user activity, patient states, and the user interfaces discussed below. A compute platform may be a single platform (e.g., at a single cloud service) or may be organized as more than one platform (e.g., among multiple distributed computing locations) that are configured to cooperate with each other. User interaction data may be coordinated with other types of medical information, including sensor data, to produce patient data. A compute platform may obtain patient data from one patient device or from among more than one patient device. Thus, for example, a therapy device such as an implanted neuromodulation device may provide some portion of the patient data, and a user device (e.g., smartphone) with an interactive user interface (e.g., provided by a smartphone app) may provide another portion of the patient data. A compute platform may also directly or indirectly obtain data from other sensor(s) and other data source(s), consistent with the following examples.

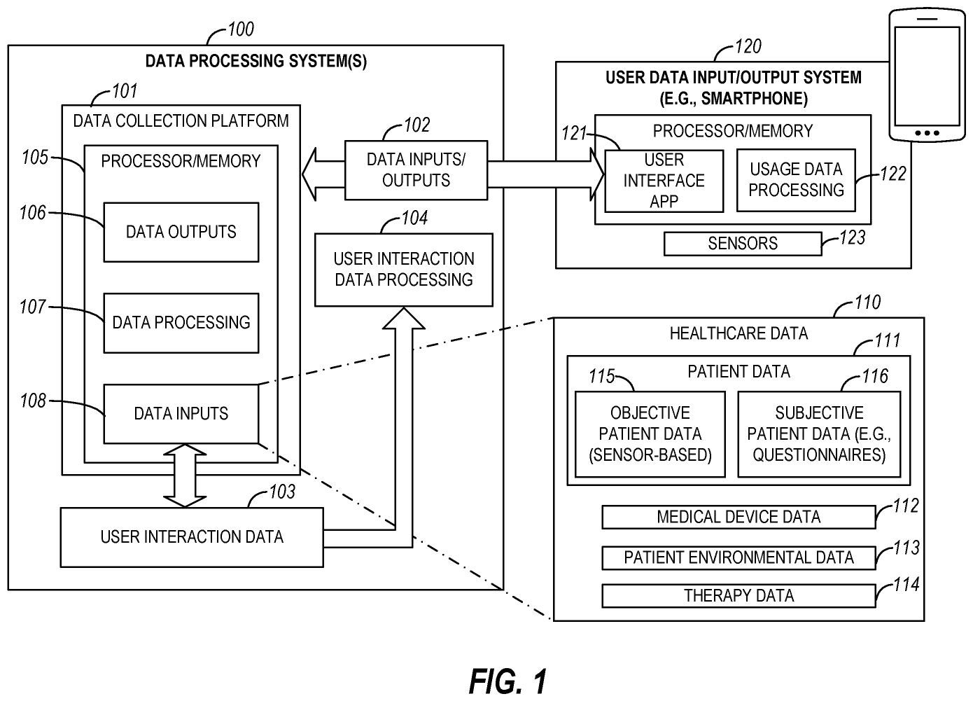

FIG. 1 illustrates, by way of example, an embodiment of a data processing system 100 configured to collect and analyze healthcare-related data in connection with the use of neurostimulation programs and user interaction workflows. The illustrated data processing system 100 is configured to include at least one data collection platform 101, which operates to collect and provide data inputs/outputs 102. The data collection platform 101 is configured to process (e.g., filter, extract, transform) the input data to produce user interaction data 103, which then can be used for one or more iterations of user interaction data processing 104. The data collection platform 101 can also generate output data in the form of recommendations, suggestions, questionnaires, and other information based on the user interaction data processing 104 or the user interaction data 103.

The data processing system 100 may be implemented at one or more server(s) or other systems remotely located from the patient. The data processing system 100 may use various network protocols to communicate and transfer data through one or more networks which may include the Internet. The data processing system 100 and data collection platform 101 may include at least one processor configured to execute instructions stored in memory (e.g., depicted as processor(s)/memory 105) to generate or evaluate data outputs 106, to obtain or evaluate data inputs 108, and to perform data processing 107 on both inputs and outputs and accompanying user interaction data. For instance, the data inputs 108 may be processed to derive or associate additional aspects (e.g., context, tags, metadata) of the user interaction data 103, such as based on the receipt of additional data from the user interface app 121, usage data processing 122, or sensors 123.

The data inputs 108 may include usage information obtained as a result of usage from the user interface app 121 or as produced by usage data processing 122, or the data inputs 108 may relate to healthcare data 110 associated with the patient or the treatment. Examples of healthcare data 110 may include patient data 111, medical device data 112, patient environmental data 113, therapy data 114, or various combinations thereof. The patient data 111 may include objective data 115 such as data collected from physiological sensor(s) and subjective data 116 such as data collected from user-answered question(s) (e.g., “How do you rate your pain?”, “What activities did you engage in today?”). The objective data 115 and subjective data 116 may be directly or indirectly produced from user interactions on the user data input/output system 120 or other patient-interactive devices.

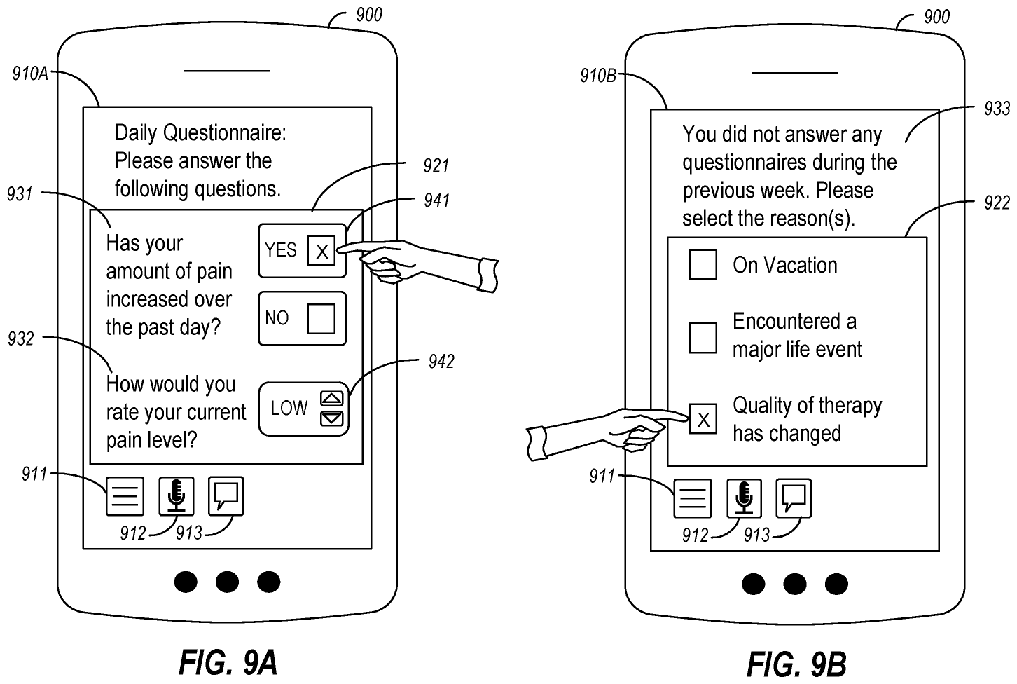

The user data input/output system 120 may be implemented at one or more devices located at or operated by the patient, such as via a smartphone, personal computer, tablet, smart home device, a remote programmer, a programming device, or another compute device or platform capable of collecting input and providing output. The user data input/output system 120 may include at least one processor configured to execute instructions stored in memory (e.g., depicted as processor(s)/memory 105) to provide or generate the data input(s) and output(s) 102 related to healthcare data and patient feedback. One such example is a user interface application 121 implemented as a graphical user interface (GUI). Examples of GUIs for data inputs and outputs include smartphone apps that are illustrated in FIGS. 9A and 9B, discussed below. Another example is usage data processing 122 which can be used to determine the usage of the user interface application 121 based on a particular time, location, or patient state.

The user data input/output system 120 may include or interface with one or more sensors 123 (e.g., location sensors such as global positioning system (GPS) sensors, activity sensors such as accelerometers, etc.) or other functionality to track, identify, determine, or derive a state of the patient or the patient activity. Other sensors such as physiological sensors may also be provided by the user data input/output system 120, to capture measurements of a patient state or patient activity directly or indirectly from the patient or from associated external systems. Examples of external system(s) include remote controls, programmers, phones, tablets, smart watches, personal computers, and the like. Thus, the user data input/output system 120 may be configured to provide all or portions of the medical device data 112, the patient environmental data 113, or the therapy data 114.

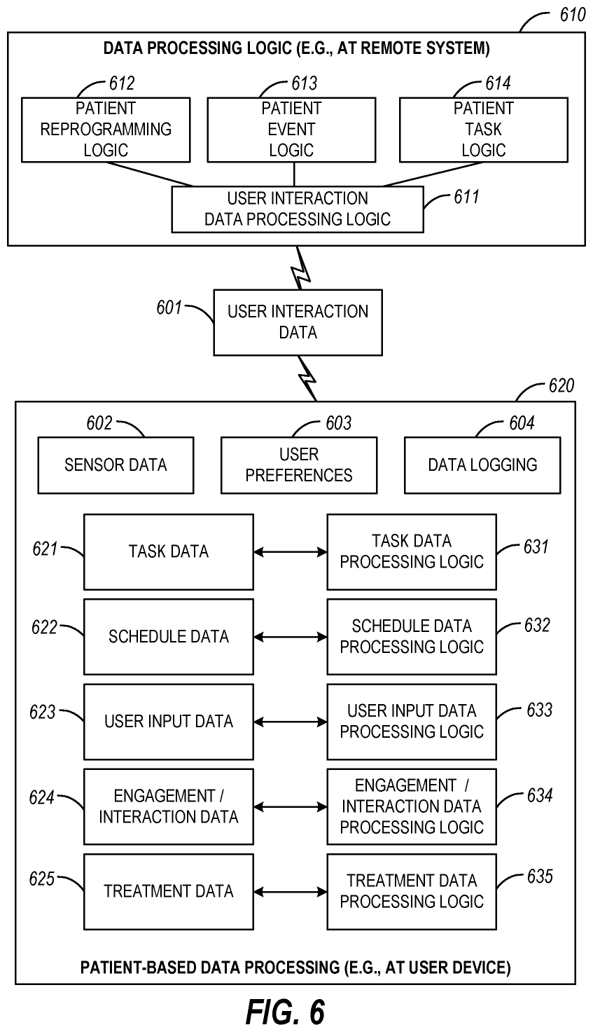

The user interaction data processing 104 operates to identify, trigger, or cause workflow actions based on the user interaction data 103, including new or modified tasks to collect information from a patient, tasks to present questions to a patient, and the like. The user interaction data processing 104 may consider other aspects of patient-specific or population data such as the healthcare data 110, sensor data, and rules and information from a variety of data sources. More details of types of user interaction data and user interaction-based data processing is discussed with reference to FIG. 6, below.

In some examples, the data collection platform 101 and the data processing system 100 may directly interface with one or more medical device(s), external system(s) or other healthcare related data source(s) to collect the healthcare data 110. One or more of the medical device(s), external system(s) or other healthcare-related data source(s) may include technology used by the data processing system 100 to collect data, and thus may form part of the data collection platform 101. Examples of medical devices include implantable and wearable devices. The medical device may be configured to only collect data, to only deliver therapy, or to both collect data and deliver therapy. The medical device may be configured to collect and provide medical device data such as device model, configuration, settings, and the like. Thus, the medical device may provide the patient data 111, the medical device data 112, the environmental data 113, and therapy data 114, particularly in specialized neurostimulation programming environments.

Other healthcare-related data source(s) may include patient data received via a provider's server that stores patient health records. For example, patients may use a patient portal to access their health records such as test results, doctor notes, prescriptions, and the like. Other healthcare-related data sources may include apps on a patient's smartphone or other computing device, or the data on a server accessed by those apps. By way of example and not limitation, this type of data may include heart rate, blood pressure, weight, and the like collected by the patient in their home. In another example, an app on a phone or patient's device may include or may be configured to access environmental data such as weather data and air quality information or location elevation data such as may be determined using cellular networks and/or GPS technologies. Weather data may include, but is not limited to, barometric pressure, temperature, sunny or cloud cover, wind speed, and the like. These and other examples of patient environmental data 113 may be correlated to the medical device data 112 or the therapy data 114, or aspects of the user interaction data 103 (e.g., so that analysis can be performed of what environment the patient was encountering when a particular answer or information was provided).

The data inputs/outputs 102 may be provided with data transferred via at least one network. The data transfer may use various network protocols to communicate and transfer data through one or more networks which may but does not necessarily include the Internet and/or various wireless networks, which may include short range wireless technology such as Bluetooth. The data may be transferred directly from at least one of the external systems and/or may be transferred directly from at least one of the medical device(s). Further, the external system(s) may be configured to receive data from an associated medical device(s) and/or receive data from other healthcare-related data source(s), and then transfer the data through the network(s) to the data receiving system(s).

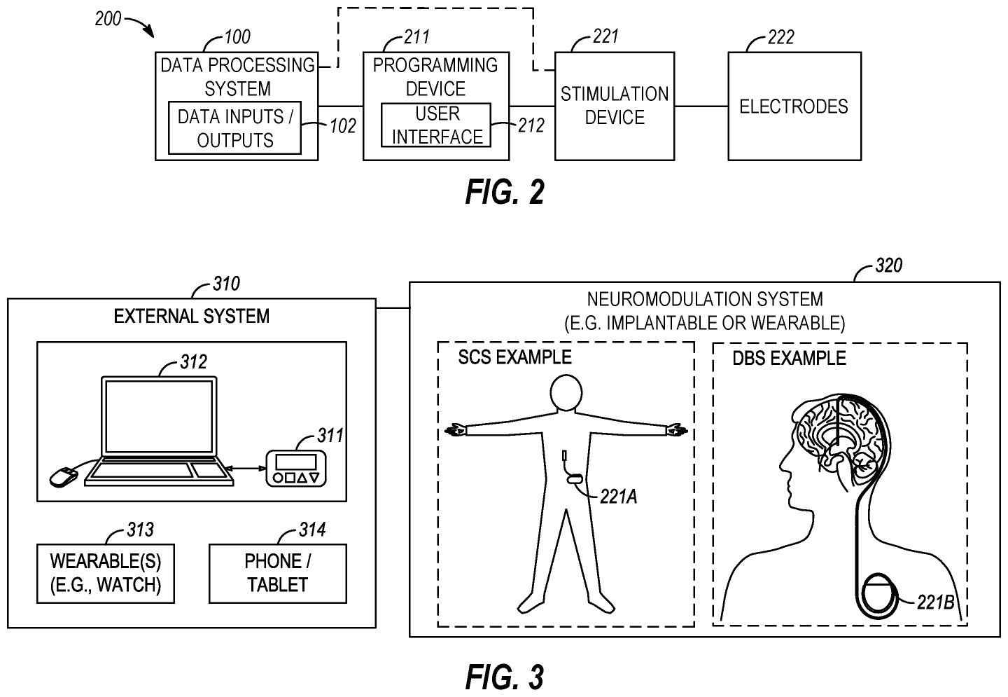

FIG. 2 illustrates a neuromodulation system as an example of a medical device system. The medical device may be configured, by way of example and not limitation, to deliver an electrical therapy using one or more electrodes 222 provided by a stimulation device 221. In the illustrated embodiment, the medical device may be a neurostimulation device, and the system may be a neuromodulation system 200.

The illustrated neuromodulation system 200 includes electrodes 222, the stimulation device 221 and a programming system such as a programming device 211. The programming system may include multiple devices. The electrodes 222 are configured to be placed on or near one or more neural targets in a patient. The stimulation device 221 is configured to be electrically connected to the electrodes 222 and deliver neuromodulation energy, such as in the form of electrical pulses, to the one or more neural targets though the electrodes 222. The system may also include sensing circuitry to sense a physiological signal, which may but does not necessarily form a part of stimulation device 221. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver, using one or more programs. The programming device 211 thus provides the user with accessibility to the user-programmable parameters. The programming device 211 may also provide the use with data indicative of the sensed physiological signal or feature(s) of the sensed physiological signal.

In various embodiments, the programming device 211 is configured to be communicatively coupled to the stimulation device 221 via a wired or wireless link. In various embodiments, the programming device 211 includes a user interface 212 such as a graphical user interface (GUI) that allows the user to set and/or adjust values of the user-programmable modulation parameters. The user interface 212 may also allow the user to view the data indicative of the sensed physiological signal or feature(s) of the sensed physiological signal and may allow the user to interact with that data. The data is provided to a data processing system 100 which provides various data inputs and outputs 102 to assist the user with the operation, configuration, maintenance, or improvement of the stimulation device 221, such as for the collection of user interaction data as discussed herein.

In some examples, the stimulation device 221 and the programming device 211 may contribute to the user interaction data 103 that is processed in the data processing system 100. The user interface 212 of the programming device 211 may also allow a user to answer a series of questions regarding the patient state (e.g., to collect patient information when used at a doctor's office, or when used at home), although other devices such as a patient smartphone may be used to obtain user interaction data and inputs as discussed below. Therapy parameters, programming selection, electrode selection, and other operational parameters may also provide healthcare-related data for use by the data processing system 100. Additional sensor(s) may also provide healthcare-related data for use by the data processing system 100.

FIG. 3 illustrates, by way of example and not limitation, the neurostimulation system of FIG. 2 implemented in a spinal cord stimulation (SCS) system or a deep brain stimulation (DBS) system. The illustrated neuromodulation system 320 connects with an external system 310 that may include at least one programming device. The illustrated external system 310 may include a clinician programmer 312 configured for use by a clinician to communicate with and program the neurostimulator, and a remote control device 311 configured for use by the patient to communicate with and program the neurostimulator. For example, the remote control device 311 may allow the patient to turn a therapy on and off and/or may allow the patient to adjust patient-programmable parameter(s) of the plurality of modulation parameters (e.g., by switching programs).

FIG. 3 further illustrates the neuromodulation system 320 as an ambulatory medical device, such as implemented by stimulation device 221A or stimulation device 221B. Examples of ambulatory devices include wearable or implantable neuromodulators. The external system 310 may include a network of computers, including computer(s) remotely located from the ambulatory medical device that are capable of communicating via one or more communication networks with the programmer 312 and/or the remote control device 311. The remotely located computer(s) and the ambulatory medical device may be configured to communicate with each other via another external device such as the programmer 312 or the remote control device 311.

The external system 310 may also include one or more wearables 313 and a portable device 314 such as a smartphone or tablet. In some examples, the wearables 313 and the portable device 314 may allow a user to obtain and provide input data, including to answer questions (e.g., on a phone/tablet screen) or to input sensor data values (e.g., from a physiologic sensor of a wearable) as part of a data collection process. In some examples, the remote control device 311 and/or the programmer 312 also may allow a user (e.g., patient and/or clinician or rep) to answer questions and provide input as part of a data collection process. The remote control device 311 and/or the programmer 312 may be used to provide other aspects of the input data, including usage data of various neurostimulation programs, events associated with such programs, location or activity events, and the like.

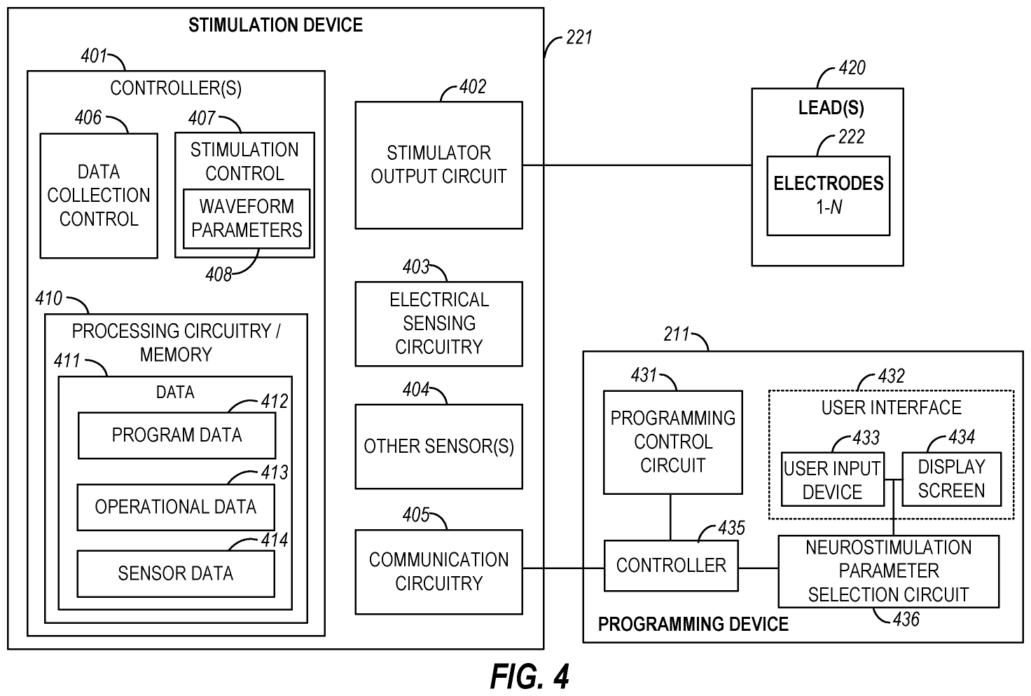

FIG. 4 illustrates, by way of example, an embodiment of a neuromodulation device, such as may be implemented as the stimulation device 221 illustrated in FIGS. 2 and 3. The stimulation device 221 may be configured to be connected to electrode(s) 222, illustrated as N electrodes, via one or more leads 420. Any one or more of the electrodes 222 may be configured for use to deliver modulation energy, sense electrical activity, or both deliver modulation energy and sense electrical activity. The stimulation device 221 may include a stimulator output circuit 402 configured to deliver modulation energy to electrode(s). The stimulator output circuit 402 may be configured with multiple (e.g., two or more) channels for delivering modulation energy, where each channel may be independently controlled with respect to other channel(s). For example, the stimulator output circuit 402 may have independent sources such as independent current sources or independent voltage sources.

In various examples, the electrodes 222 may include a stimulation electrode or a sensing electrode. The stimulation electrode is configured for use in delivering modulation energy, and the sensing electrode is configured for use in sensing electrical activity. As illustrated, the stimulation electrode may also be used in sensing electrical activity, and the sensing electrode may also be used in delivering modulation energy. Thus, the term “stimulation electrode” does not necessary exclude the electrode from also being used to sense electrical activity; and the term “sensing electrode” does not necessarily exclude the electrode from also being used to deliver modulation energy.

The stimulation device 221 may include electrical sensing circuitry 403 configured to receive sensed electrical energy from the electrode(s), such as may be used to sense electrical activity in neural tissue or muscle tissue. The sensing circuitry may be configured to process signals in multiple (e.g., two or more) channels. By way of example and not limitation, the electrical sensing circuitry 403 may be configured to amplify and filter the signal(s) in the channel(s).

The controller 401 may be configured to detect one or more features in the sensed signals. Examples of features that may be detected include peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline. Detected feature(s) may be fed into a control algorithm, which may use relationship(s) between the feature(s) and waveform parameter(s) to determine feedback for closed-loop control of the therapy. Some embodiments of the stimulation device 221 may include or be configured to receive data from other sensor(s) 404. The other sensor(s) 404 may include physiological sensor(s), environmental sensor(s), or proximity sensor(s).

The stimulation device 221 may include a controller 401 operably connected to the stimulator output circuit 402 and the electrical sensing circuitry 403. The controller 401 may include a stimulation control 407 (e.g., logic) configured for controlling the stimulator output circuit 402. For example, the stimulation control 407 may include start/stop information for the stimulation and/or may include relative timing information between stimulation channels. The stimulation control 407 may include waveform parameters 408 (e.g., associated with a program or a defined set of parameters) that control the waveform characteristics of the waveform produced by the stimulator output circuit 402. The waveform parameters 408 may include, by way of example and not limitation, amplitude, frequency, and pulse width parameters. The waveform parameters 408 may include, by way of example and not limitation, regular patterns such as patterns regularly repeat with same pulse-to-pulse interval and./or irregular patterns of pulses such as patterns with variable pulse-to-pulse intervals. The waveform parameters may, but do not necessary, define more than one waveform shape (e.g., including a shape other than square pulses with different widths or amplitudes). The stimulation control 407 may be configured to change waveform parameter(s) (e.g., one or more waveform parameters) in response to user input and/or automatically in response to feedback.

The controller 401 may include a data collection control 406 configured for use by the stimulation device 221, and the data collection platform 101 of a data processing system 100 (see FIGS. 1-2), to collect healthcare related data. The controller 401 may include a processor and/or memory 410 (e.g., with instructions) for use to control the data collection using the data collection control 406 and control the stimulation via the stimulation control 407. The memory may also provide storage for storing different types of collected healthcare-related data 411, such as program data 412, operational data 413, sensor data 414, and the like. Examples of sensor data 414 collected by the stimulation device 221 or other devices discussed herein may include, by way of example and not limitation, heart rate, heart rate variability, oxygen saturation, activity, posture, steps, gait, temperature, evoked compound action potentials (ECAPS), electromyograms (EMGs), electroencephalograms (EEGs), weight, blood pressure, and the like. Examples of program data 412 may include, by way of example and not limitation, stimulation settings such as amplitude, pulse width, pulse frequency period, duration of burst of pulses, active electrodes, electrode fractionalization controlling the distribution of energy (e.g., current) to active electrodes, waveforms, pulse patterns including various complex patterns, and the like. Examples of operational data 413 of the stimulation device 221 may include, by way of example and not limitation, electrode-tissue impedance, fault conditions, battery information such as battery health, battery life, voltage, charge state, charging history if rechargeable, MRI status, Bluetooth connection logs, connection with Clinician Programmer, hours of operation/duration of implant, and the like. Other device information may include device model and lead model.

The neuromodulation device may include communication circuitry 405 configured for use to communicate with other device(s) such as a programming device 211, remote control, phone, tablet and the like. The healthcare-related data may be transferred out from the neuromodulation device for transfer to a data processing system, as discussed above. As shown, a programming device 211 includes a programming control circuit 431, a user interface 432 (e.g., including a user input device 433 such as a buttons and a display screen 434), a controller 435, and other components (e.g., an external communication device, not shown) to effect programming of a connected neurostimulation device. The operation of the neurostimulation parameter selection circuit 436 enables selection, modification, and implementation of a particular set of parameters or settings for neurostimulation programming (e.g., via selection of a program, specification by a closed-loop or open-loop programming process, specification by a patient or clinician, or the like).

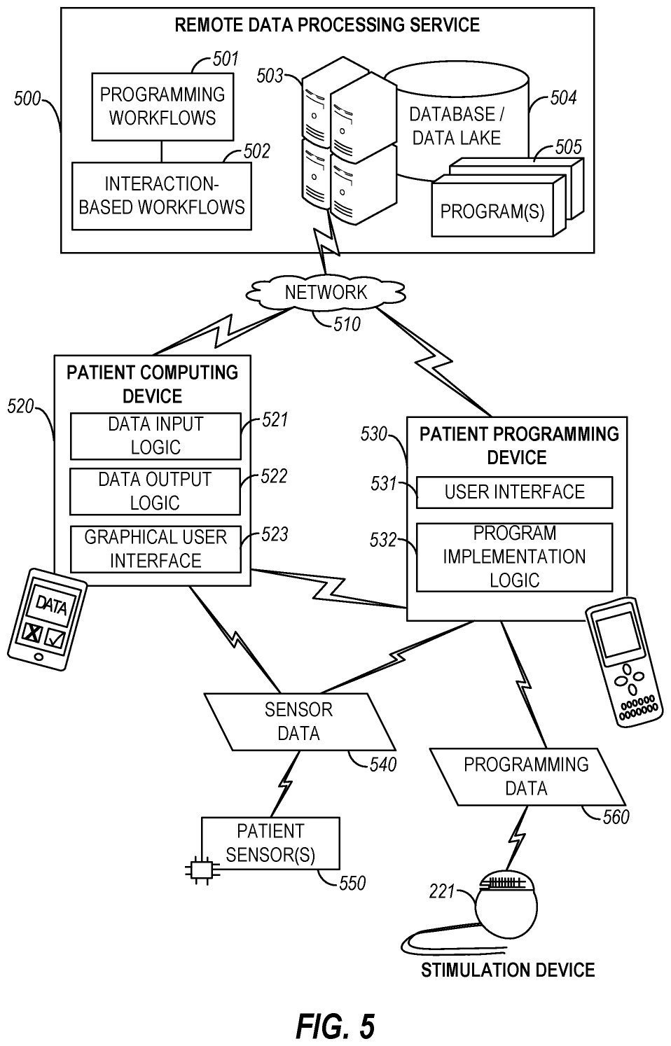

FIG. 5 illustrates, by way of example, an embodiment of a data processing system implemented as a remote data processing service 500, which coordinates with patient use of a stimulation device 221 and various user interaction and task-based workflows. At a high level, a remote data processing service 500 uses algorithms, logic, and other implementations of interaction-based workflows 502 and neurostimulation programming workflows 501 to collect and analyze patient data (e.g., feedback, activity, task results, etc.) collected on an ongoing basis. Such patient data may be hosted in a database 504 or another large-scale data store (e.g. data lake) for the patient or a population of patients, and processed by compute hardware 503 of one or more computing systems.

The remote data processing service 500 can be used to perform a variety of data-driven operations, including using the interaction-based workflows 502 to collect additional event or activity information from the patient (e.g., what program was being used by the stimulation device 221 when a patient provided particular feedback). The remote data processing service 500 may also use the programming workflows 501 to generate interaction-driven programming settings and parameters (e.g., in one or more programs 505) that are customized to the human patient. For instance, the remote data processing service 500 may employ the compute hardware 503 as part of the programming workflows 501 or interaction-based workflows 502 to generate or control questionnaires, interaction tasks, diagnostic actions, informative alerts, or programming recommendations or actions. The programming settings and parameters may be implemented automatically or manually on the stimulation device 221 or via a patient computing device 520 or patient programming device 530.

The remote data processing service 500, in the depicted example, communicates with one or both of the patient computing and programming devices 520, 530, to obtain patient data (e.g., that includes or is associated with interaction data). The remote data processing service 500 may also include data analysis or processing engines (not shown) that parse and determine a state of a particular patient from inputs and correlate program usage to an activity, task, or event (e.g., to determine what programs and programming settings are beneficial or not beneficial to the patient, when the patient provides certain feedback). In some examples, the state of treatment may be based on correlating the historical use of a neurostimulation program or set of parameters with a state of a patient (e.g., identifying that a pain condition became worse or better after beginning use of a particular program) and activities or characteristics associated with use of a particular program.

The remote data processing service 500 may also derive and analyze a variety of forms of patient input and patient data related to a neurostimulation program or neurostimulation programming parameters. For instance, the remote data processing service 500 may receive information from program usage, questionnaire selections, or even freeform text or audio input originating from a human patient, via the patient computing and programming devices 520, 530. In addition to providing recommended programs, the remote data processing service 500 may provide therapy content and usage recommendations to the patient computing and programming devices 520, 530, in response to the inputs.

A patient may enter input data that is related to an assigned interaction task, past or planned activities, or other events via the patient computing device 520 or the patient programming device 530. Additional detail of how input data is collected is discussed with reference to the data processing logic and user interfaces discussed in more detail below. In an example, the patient computing device 520 is a computing device (e.g., personal computer, tablet, smartphone) or other form of user-interactive device that receives and provides interaction with a patient using a graphical user interface 523. The patient computing device 520 may include data input logic 521 and data output logic 522 to control various interaction functions. For instance, the data input logic 521 may receive and ask for input from a patient via questionnaires, surveys, messages, or other inputs. The inputs may provide text related to pain or satisfaction, for example, which can be used to identify a psychological or physiological state of the patient, neurostimulation treatment results, or related conditions, including those associated with a particular task, location, event, activity, program, etc.

A patient programming device 530 is depicted as including a user interface 531 and program implementation logic 532. The program implementation logic 532 may provide the patient with the ability to implement or switch to particular neurostimulation (including those programs 505 generated or updated by remote data processing service 500). The patient programming device 530 may be used for closed-loop programming such as with the receipt of instructions, recommendations, or feedback (including clinician recommendations, behavioral modifications, etc., selected for the patient) that are automatically selected based on detected conditions.

The remote data processing service 500 may also utilize sensor data 540 from one or more patient sensors 550 (e.g., wearables, sleep trackers, motion tracker, implantable devices, etc.) among one or more internal or external devices. The sensor data 540 may be used to determine a customized and current state of the patient condition or neurostimulation treatment results, including those tracked or associated with a particular task, activity, event, or location. In various examples, the stimulation device 221 also includes sensors which contribute to the sensor data 540 to be evaluated by the remote data processing service 500.

In an example, the patient sensors 550 are physiological or biopsychosocial sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

FIG. 6 illustrates data types and processing logic associated with the data input or output examples discussed for user interaction data processing. The data processing logic 610 (e.g., implemented by data processing system 100, remote data processing service 500, or other systems discussed above) may operate to perform data-driven actions and workflows, with the use of user interaction data processing logic 611. The user interaction data processing logic 611 may be used to trigger particular interaction or treatment workflows involving a patient, based on particular conditions associated with tasks, events, activities, or locations, consistent with the examples discussed herein.

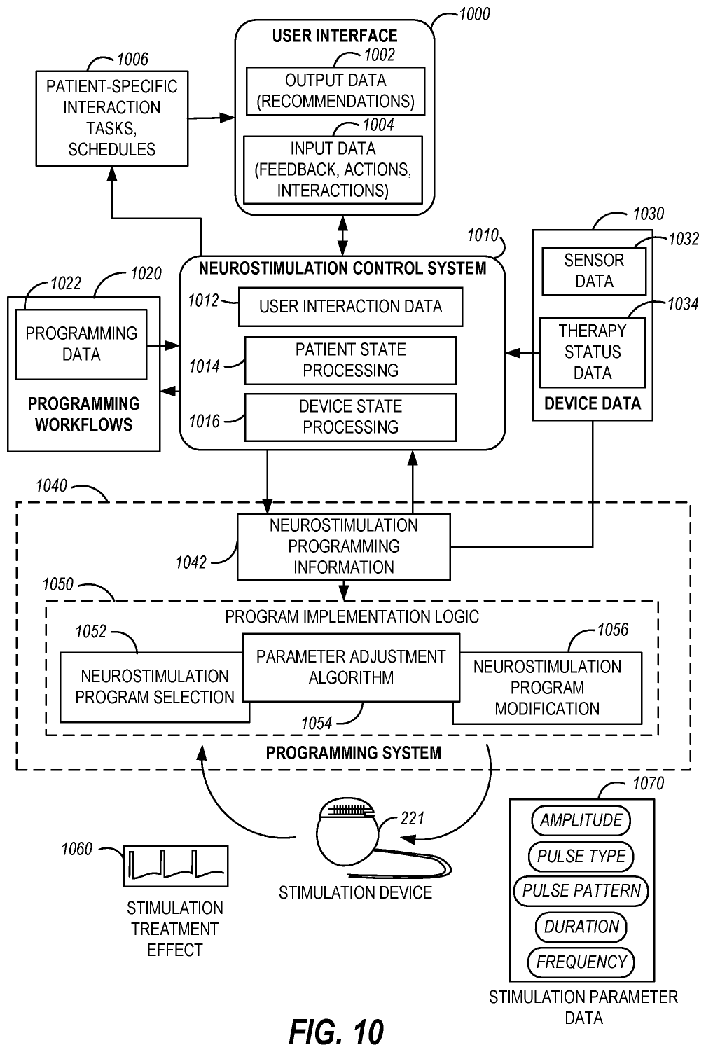

The data processing logic 610 depicts three types of workflows that can involve the patient. This includes: a workflow for patient reprogramming logic 612, which may enable recommended or automatic device reprogramming based on user interaction and detected patient states; a workflow for patient event logic 613, which may cause particular event logging, alerts, notifications, or data analysis based on one or more patient activities or patient states that are detected as a result of user interaction; and a workflow involving patient task logic 614, which may provide customized questions, surveys, or other interactive content (inputs and outputs) to be assigned to a particular patient to collect interaction data. In still further examples, the patient reprogramming logic 612 may enable closed-loop programming. Further details on an implementation of closed-loop programming is provided with reference to FIG. 10, discussed in more detail below. Other workflows involving a caregiver (e.g., a family member, personal nurse, trusted friend, etc.) or clinician (e.g., doctor, nurse, medical device company representative, pharmacist, or another medical professional) may be provided by the user interaction data processing logic 611 or other logic elements not shown. Various aspects of artificial intelligence, machine learning, and data-driven logic (not depicted) may be implemented in any of the components of the data processing logic 610.

FIG. 6 also shows various types of patient-based data processing 620 of user interactions, such as data processing that may be ongoing at a user computing device (e.g., patient computing device 520, and software implemented thereon) as new user interactions are requested and received. Such data processing may include the collection or evaluation of sensor data 602, the performance of data logging 604 to collect user interaction data (e.g., logged data that is communicated at regular intervals to a data processing service), and the ongoing collection and coordination of task data 621, schedule data 622, user input data 623, engagement or interaction data 624, and treatment data 625. The collection of the sensor data 602 and other types of user interaction data may be controlled based on user preferences 603, which may include privacy settings or permissions.

In specific examples, user interaction tasks and events may be processed by the data processing 620, to track and respond to particular attributes of user interactions that occur at a user device. Example data collection and processing operations that may occur at the user device may include: task data 621 that is processed by task processing logic 631 to perform particular user interaction tasks and track task properties; schedule data 622 that is processed by schedule data processing logic 632 to deliver user interaction tasks at particular times or dates; user input data 623 that is processed by user input data processing logic 633 to refine or track user selections and inputs (e.g., questionnaire question and answer selections, freeform text, etc.); engagement and interaction data 624 that is processed by engagement and interaction data processing logic 634 to track overall engagement and user interaction events; treatment data 625 that is processed by treatment data processing logic 635 to track the state of a neurostimulation treatment associated with the patient user. Other data may be stored and/or transferred from the user device, including event data to track the occurrence of events, contextual data, time data, and the like.

Other types of objective data and subjective data may be collected or processed by the data processing 620, including data measurements of a patient that are not directly obtained from a user interaction. Objective data, as used herein, is data that can be obtained from a measurement or direct observation. Objective data may be measured by a sensor and may be provided via user input when the user has access to objectively determined information. Categories of objective data may include physiological parameter data, therapy data, device data, and environmental data. By way of example and not limitation, physical parameter data may include data such as: heart rate, blood pressure, respiration rate, activity, posture, electromyograms (EMGs), neural responses such as evoked compound action potentials (ecaps), glucose measurements, oxygen levels body temperature, oxygen saturation and gait. By way of example and not limitation, therapy data may include: neuromodulation programs, therapy on/off schedule, dosing, neuromodulation parameters such as waveform, frequency, amplitude, pulse width, period, therapy usage and therapy type. By way of example and not limitation, device data may include: battery information (voltage, charge state, charging history if rechargeable), impedance data, faults, device model, lead models, MRI status, Bluetooth connection logs, connection history with a Clinician's Programmer (CP). By way of example and not limitation, environmental data may include: temperature, air quality, pressure, location, altitude, sunny, cloudy, precipitation, etc. Subjective data can include information received from the patient or another human user (e.g., caregiver, clinician, etc.). For example, the patient's quantification of pain can provide subjective data.

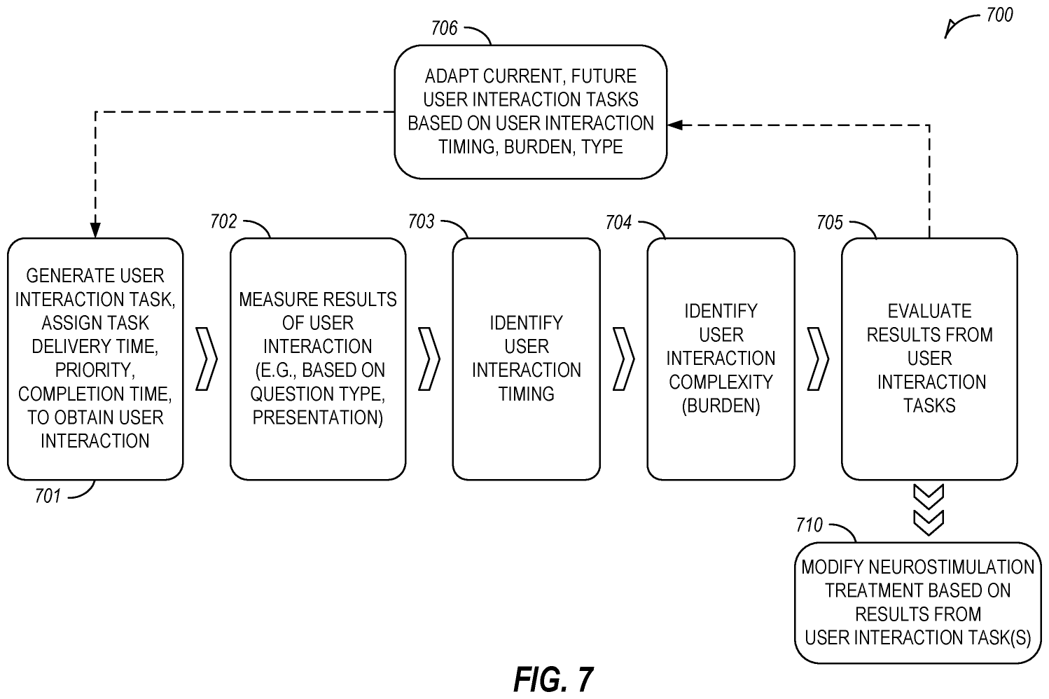

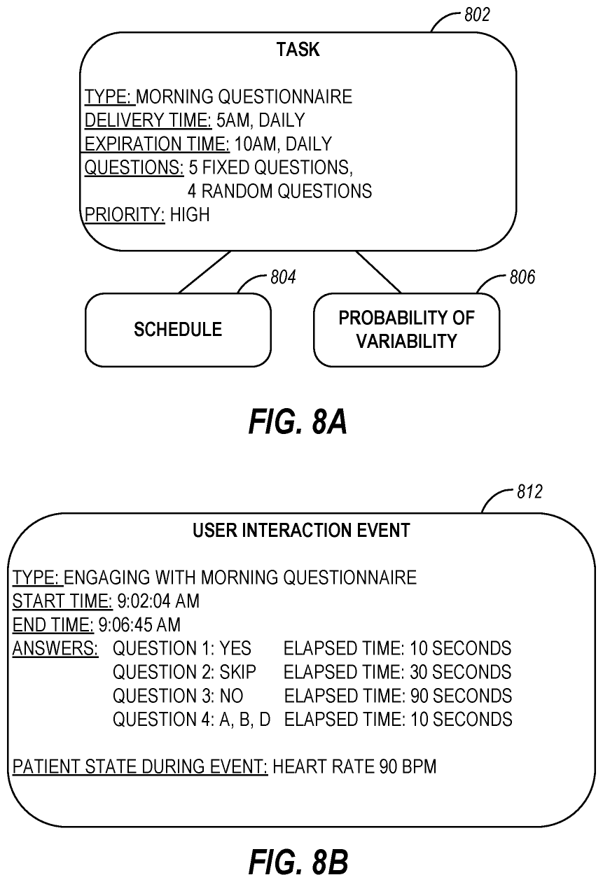

From the above-described data and derived information, and data flows, user interaction data 601 can be used to trigger various workflows, including those related to additional user interaction tasks, user interaction events, or patient reprogramming. To demonstrate these use cases, FIG. 7 depicts an adaptive workflow for the collection and evaluation of user interaction data, FIGS. 8A and 8B depict data properties associated with tasks and user interaction events, FIGS. 9A and 9B depict user interfaces for collecting user interaction data, and FIG. 10 depicts workflows for adaptive neurostimulation programming.

FIG. 7 depicts a workflow 700 for the generation, delivery, and evaluation of user interaction events. This workflow 700 specifically demonstrates an iterative process of user interaction task creation and assignment, measurements of attributes of user interaction, and adapting subsequent user interaction tasks and workflows to the attributes of user interaction. In the following, adaptations to user interaction tasks can be based on timing, complexity (i.e., burden on the user), and the need for specific user input (e.g., answers to questions).

At 701, user interaction tasks are generated and are assigned with task properties. In an example, tasks are assigned with defined delivery times, and associated questions are assigned a priority, anticipated completion times, and probability of variability. These task assignments may be associated with expected user patterns of interaction. When a user interaction task is created, the task can be assigned to be delivered and expire at set times that are aligned to a predicted general patient's schedule. For example, a morning questionnaire may be scheduled to arrive at 5 am and expire at 10 am.

At 702, the results of the user interaction are measured. For instance, data is collected to objectively measure the user's pattern or habit of opening/engaging with a questionnaire in the presenting software app. The measurement of user interaction may include an evaluation of factors such as: When does the patient open the application, or when does the patient rarely/never open the application (particular days and time); When sent a task, how long is it until the user logs into the app and starts the task?; What type of notification was provided to the user for the task? How much time elapsed after the user first opened the app until receiving the task, and how long was it until the user started the task, completed 50% of the task, completed 80% of the task, or finished the task? How many tasks does the user complete in a session? How many questions does the user complete in a session? How long does the user stay engaged in a session? In these contexts, a user interface session can be tracked based on a set level of interaction with the app (e.g., being logged into the app, being away from the app screen for no more than a specific number of minutes, or the like). User interactions can be measured using data events embedded into a client software application, or recorded at a server in a database or data lake. Granular user interaction events that are tracked can include, for instance, ‘Started Task’, ‘Completed Task’, ‘Answered Question’, ‘Opened Tab in App’, ‘Started App Session’, etc.

At 703, user interaction timing is identified. This may include a detailed analysis of how long that a patient viewed a particular screen or item and then provided (or did not provide) user interaction. An extended time for response may indicate confusion or difficult questions, whereas a short time for response may indicate that the user is not reading the question (e.g., because the user has seen it several times and is familiar with it). A per-item (e.g., per question) time measurement can be used to detect scenarios where the view time exceeds some threshold, or exceeds a change threshold in view time per-item, to trigger some action. This action may include the presentation of a related event, such as a ‘Do you need help with this question?’ screen. Based on timing, the formulation of the question may change to trigger the user's attention and prevent a user from improperly answering the question too fast. In a similar fashion, an analysis may be performed to determine whether the user has skipped the same question repeatedly, or marked a question as not applicable. Such user interaction may indicate that the user does not understand, cannot answer, or is uncomfortable with the question. Thus, related user interaction events may be logged to detect when certain behaviors exceed a ‘threshold frequency’ of ‘skip’ behavior, to then perform further analysis or request feedback to understand the behavior. Similarly, analysis may be performed when if a user frequently marks “other” to a multiple choice question and enters text manually, provides similar or identical text entries for multiple questions, or answers a question consistently for a question that has high variability (e.g., showing that the user may have habituated to the question and may not be thinking about the question when they answer it).

At 704, a user interaction burden (e.g., complexity or difficulty) for a particular task is identified. The user interaction burden can be tailored to the patient's estimated willingness to complete multiple tasks, complete a certain number of questions, stay engaged for a set amount of time, or a combination of the above. For instance, a particular user may be willing to complete multiple tasks but may stop answering after they have completed a certain number of questions or after a certain time has elapsed. A different user, in contrast, may log in and complete only one task at any given time despite differences in task length. For the first example, tasks could be presented such that only a few questions below the patient's maximum answering threshold are ever presented at any given time. In the second example, tasks can be reformatted into a single, longer task rather than multiple shorter ones. For users that are easily overwhelmed, multiple tasks and the associated questions could be reduced such that a single task is sent every day that contains no more than a defined number of questions (e.g., 5 questions maximum). Other variations may include tasks that are normally sent every day to be reduced to a few times a week to allow for the collection of other critical tasks. Questions within each task could be randomized and shuffled intelligently to maximize the probability of collecting required information. For questions that schedule a full data collection at the end of a week, the schedule may be changed to receive all data by an end of month. Lower priority tasks/questions may be dropped entirely.

At 705, the results of the user interaction tasks are evaluated. Even the best users are unlikely to answer questions and engage with interaction tasks every day. In such scenarios, predictive algorithms can be used to derive what a user's response most likely would have been when a data point is missing, by using data imputation or model-based data prediction. In some cases, data is needed to inform various algorithms, so a question's priority can adapt to reflect a need for the information from a clinician, an algorithm, etc. Additional actions can be triggered if a user has not answered a required question too many times. This could also occur because critical timepoints require specific information more than others that cannot be predicted or replaced. An imputation or prediction algorithm can be ‘data hungry’ for that particular data type (e.g., sleep duration), and as such, that data type is assigned a higher priority and can edge out other data types in the upcoming questionnaire.

At 706, current or future user interaction tasks are adapted, such as for use in a subsequent user interaction workflow. For example, timing of tasks may be adapted to coincide with a patient's observed schedule and pattern of engagement with the app. As a first example: consider a scenario where a patient only opens their phone between 7-8 am when they wake up; here, future user interaction tasks may be scheduled to provide notifications in that time range, or a window of time where tasks would have been scheduled to fire in that window are collapsed to fire in the ‘active’ window of 7-8 am. As a second example, consider a scenario where a patient never interacts with the application on a specific day, like a Saturday, but some tasks are anticipating an interaction rate of once a day 7 days a week. In this scenario, on each day of the week a different subset of information is requested, so that by the end of the week all data points are collected (and, at a lower patient burden then asking all missing questions in a day). The order of the questions may be randomized from week to week to prevent consistent data loss from such a patient as exampled above. As a similar example, once enough data has been collected to predict a trend that the patient has a low probability of logging into the application on Saturday, the patient's tasks which are anticipating a 7 day a week interaction could be shifted to a 6 day a week interaction. This adapts the workflow to push more questions each day while also only pushing out these questionnaires on the days when the patient interacts with the application. As a yet another example, tasks generally scheduled for certain times (such as the morning) could be shifted based on the patient's interactions (for example, for a user who is active in the morning versus a user who is active in the evening).

Other aspects of timing of current or future user interaction tasks may also be modified. Questions can be assigned with an anticipated interaction time (e.g., a total user average and current user average). Questions where a user lingers excessively or frequently skips could be replaced with a similar question or several questions of different wording/less complexity. If questions are still reviewed extensively or skipped, and the questions are gauged to be simple, the user may be uncomfortable answering the question. Additional questions could be included to clarify this, or the question could be removed entirely. If a user is answering the same questions quickly and questions are not achieving the variability anticipated, question order, answer order, question type, and answer type could all be varied such that the same information is meant to be collected but is now presented in a unique manner. Answer objects and an order for a given question can be tailored to reflect a user's previous answers.

In further examples, questions may be modified to provide an auto-completed or auto-filled answer. This may apply, for example, to questions where the user has repeatedly marked “other” and provided a consistent answer on manual entry (e.g., which can be provided in an auto fill option containing the text information that the user usually writes). Such answers may be filled instantly, or once the user has typed a set amount of similar characters. Likewise, for questions where the user has repeatedly marked “other” and indicated the same answer, a custom answer object may be provided to select the previous answer via methods such as multiple choice or drop down selections. This type of a custom answer object is unique to the individual and would not be shown to another user.

Other types of measuring and adapting may occur for changes in defined user interaction behavior. After sufficient time, the patient's behaviors should be well defined, and this definition can be saved for the patient. However, a patient's pattern of behavior should not be assumed to remain static. Thus, the same measurements utilized to initially derive a patient's behavior can be continually run to identify changes in patient behavior. A significant change in behavior associated with the previously defined variables, could lead the application to send specific tasks (e.g., Are you on vacation? Is there a major life event? Has the quality of your therapy sufficiently changed?). The user interaction workflows may adapt (as described previously) to the patient's new patterns of behavior after a set time.

At 710, the results of the user interaction may be used to modify a neurostimulation treatment. The modification of the neurostimulation treatment may be coordinated with data from other sensors, devices, and software applications, including those which measure user interaction behaviors or activities. Other adaptations of user interaction tasks and workflows (e.g., at 706) may be coordinated with the neurostimulation treatment. In further examples, multiple apps and devices (e.g., from the same patient or multiple patients) share interaction data to a data processing service, with the purpose of maximizing engagement and shifting user focus to critical engagement needs, including for a device manufacturer or clinical service provider. For instance, a priority of interaction can be established across user apps so that a remote programmer app or device can prioritize presentation of an important status message to the user (e.g., that their battery is critically low) over other scheduled tasks/questions. Likewise, time of usage of a user interaction application (to provide surveys and patient state information) can drive attention to another app, device, or related product. Similar approaches may be used for users who engage regularly with a wearable device, but not a mobile app, including having tasks, questions, and notifications sent to the wearable rather than a mobile device (with questions selected and prioritized that present best in this format). In turn, the wearable device can be used to inform the user that they need to return to their mobile app in order to review a new stimulation setting or program, etc.

FIG. 8A depicts example data properties associated with a user interaction task, such as a task assigned to a patient to complete a questionnaire. In the depicted example, a task 802 is assigned with properties such as a type, delivery time, expiration time, question contents, and a priority. The task 802 may be associated with additional schedule data 804 (e.g., an anticipated completion time (e.g., daily or weekly)), and a probability of variance or variability 806. As an example, a user interaction task can be scheduled to be delivered daily and ask the same ten questions every day. However, some of the questions may be rated as more important than others, or some tasks could be scheduled daily to ask a defined set random (e.g., four non-repeating) questions from a list of questions (e.g., 28 available questions), daily. Then, at the end of the week, on average, all questions would be answered. The probability of variance or variability 806 may be used because some questions anticipate a high degree of fluctuation while others are predicted to be more stable. For example, consider a scenario where a patient has no tremor but has bradykinesia and known fluctuations in therapy. In this scenario it is expected that recorded tremor symptoms will be consistent while bradykinesia scores will fluctuate frequently.

FIG. 8B likewise depicts data properties included as part of a recorded user interaction event 812. For instance, the user interaction event 812 may be used to represent the outcome of a questionnaire response from a patient that provides data values. In the depicted example, the user interaction event 812 includes information on the type of user interaction, a start and end time of the user interaction event, time information on individual questions and answers, individual answers to provided questions (including an indication of skipped questions), patent state information collected during the event. Additional data values may be recorded depending on the type of user interaction scenario and available data.

FIGS. 9A and 9B depict data input user interfaces 910A, 910B (e.g., a smartphone app graphical user interface) which provide output on a user device 900 (e.g., a smartphone). In various examples, a patient's phone, computer, tablet, or programming remote control (or, multiple of these devices) may provide such user interfaces. The user interfaces 910A, 910B are also depicted to include graphical touchscreen interactivity, but it will be understood that other user input functionality, such as menu selection functionality 911, voice input functionality 912, or freeform text input functionality 913 (including non-graphical inputs such as voice interactions controlled by a chatbot or by virtual assistants or agents such as Amazon® Alexa, Google® Assistant, Apple® Siri, etc.) may be provided.

FIG. 9A specifically depicts the data input user interface 910A as including functionality to present a daily questionnaire, with a set of one or more questions 921. This may include the presentation of a first question 931 with a binary selection 941 (e.g., yes or no), the presentation of a second question 932 with a value selection (e.g., a value from among multiple options). FIG. 9B similarly depicts the data input user interface 910B as including a set of one or more questions 922 with multiple choice selections. Other types of user interface controls may be implemented. Additionally, instructive outputs, such as a message 933 which relates to user interaction trends, may be provided to the user.

FIG. 10 illustrates, by way of example, an embodiment of a data processing flow for controlling the neurostimulation treatment of a human patient, which may occur based on the user interaction data processing operations discussed above. Specifically, this data processing flow shows how a neurostimulation control system 1010 may perform patient state processing 1014 and device state processing 1016 functions, based on user interaction data 1012 (discussed above) that indicates a patient state, events, activities, or relevant conditions. Here, as the patient state processing 1014 is used to determine a patient state and the device state processing 1016 is used to determine a device state, various improvements to neurostimulation programming can be provided (e.g., with programming workflows 1020), which in turn produces updated programming data 1022 for deployment with new neurostimulation programming information 1042).

In this example, the user interface 1000 provides output data 1002 (e.g., recommendations, questions, etc.) that help collect user interaction data 1012 related to the state of the patient and the usage and efficacy of neurostimulation programs. The user interface 1000 collects input data 1004 (e.g., answers and other feedback data, commands, other user interactions), as discussed above, relating to medical conditions or the use of one or more neurostimulation programs. The input data 1004 may be collected based on the use of patient-specific interaction tasks and schedules 1006, which are driven from an interaction workflow as discussed herein.

FIG. 10 also depicts the evaluation of device data 1030, such as sensor data 1032, therapy status data 1034, and other treatment aspects which may be obtained or derived from the stimulation device 221 or related neurostimulation programming and device operation. The device data 1030 and the inputs received with the user interface 1000 allow relevant user interaction data 1012 to be collected, which is evaluated during patient state processing functions 1014 and device state processing functions 1016. Various programming workflows 1020 generate new or updated programming data 1022 (e.g., programs, program parameter values) based on some combination of the user interaction data 1012, and from results of the patient state processing functions 1014 and device state processing functions 1016.

The remainder of the data processing flow illustrates how the neurostimulation control system 1010 implements programming, such as in a closed loop (or partially-closed-loop) system. A programming system 1040 uses programing information 1042 provided from the neurostimulation control system 1010 as an input to program implementation logic 1050. This may be affected, in part, by device data 1030 including sensor data 1032 and therapy status data 1034. The program implementation logic 1050 may be implemented by a parameter adjustment algorithm 1054, which affects a neurostimulation program selection 1052 or a neurostimulation program modification 1056. For instance, some parameter changes may be implemented by a simple modification to a program operation; other parameter changes may require a new program to be deployed. The results of the parameter or program changes or selection provides various stimulation parameters 1070 to the stimulation device 221, causing a different or new stimulation treatment effect 1060.

By way of example, operational parameters of the stimulation device which may be generated, identified, or evaluated by the neurostimulation control system 1010 may include amplitude, frequency, duration, pulse width, pulse type, patterns of neurostimulation pulses, waveforms in the patterns of pulses, schedule (including on-off cycles of one or more stimulation settings), and like settings with respect to the intensity, type, location, and timing of neurostimulator output on individual or a plurality of respective leads. The neurostimulator may use current or voltage sources to provide the neurostimulator output, and apply any number of control techniques to modify the electrical simulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

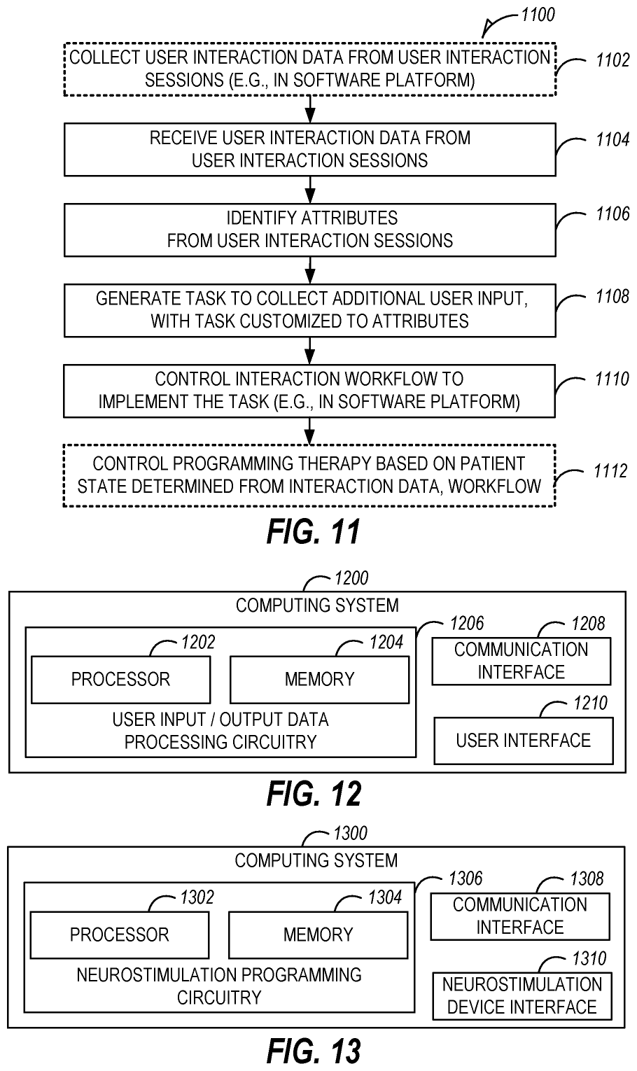

FIG. 11 illustrates, by way of example, an embodiment of a processing method 1100 implemented by a system or device to analyze user interaction activities in connection with a neurostimulation treatment, e.g., in connection with various user interaction data and workflows collected with the scenarios above. For example, the processing method 1100 can be embodied by electronic operations performed by one or more computing systems or devices (including those at a network-accessible remote service) that are specially programmed to implement data analysis and/or neurostimulation data processing operations. In specific examples, the operations of the method 1100 may be implemented through the systems and data flows depicted above, at a single entity or at multiple locations, including with coordination of patient, caregiver, or clinician devices.

In an example, the method 1100 begins at 1102 in response to the use of a computing device, to collect user interaction data from one or more user interaction sessions (e.g., historical interaction sessions occurring at prior time(s), or in an ongoing session) occurring with a software platform. This user interaction data is associated with a patient undergoing the neurostimulation treatment, and such user interaction data may be provided by the patient, a caregiver, or a clinician, as noted above. In an example, the computing device is a patient computing device provided by a personal computer, tablet, smartphone, remote control, or wearable device, which implements the software platform (e.g., the applications and user interfaces discussed above) to query the user and conduct the user interaction sessions. The user interaction sessions may be conducted within an interaction workflow that defines the order, type, and amount of interactions. In an example, the interaction workflow is a patient interaction workflow to occur directly with the patient (e.g., to collect data from the patient user directly about his or her treatment, medical condition, and state). In other examples, the interaction workflow is a caregiver workflow to occur with a caregiver associated with the patient or a clinician workflow to occur with a clinician associated with the patient (e.g., to collect data from the caregiver or the clinician, to query about the status or condition of the patient).

The method 1100 continues at 1104 by obtaining or receiving user interaction data from the one or more user interaction sessions. In an example, the user interaction data indicates attributes of one or more user interactions in a software platform that collect user feedback relating to the neurostimulation treatment. For instance, the user feedback may be collected from the patient using one or more questions during each session of the one or more user interactions, as the one or more answers (corresponding to the one or more questions) provide details on a state of the patient undergoing the neurostimulation treatment.

The method 1100 continues at 1106 by identifying attributes from the one or more user interaction sessions, from processing of the user interaction data. In an example, the attributes of the user interactions may relate to at least one of: a number of tasks completed in each session; a number of questions completed in each session; an ongoing or historical usage of the software platform; an ongoing or historical pattern of use of the software platform; an amount of time of engagement in each session; an amount of time of engagement with respective types of questions in each session; a time of day of engagement in each session; or a pattern of engagement in each session.

The method 1100 continues at 1108 by generating a task to collect additional user input, by creating such a task to be customized to the patient based on the identified attributes of the one or more user interactions. For example, the task includes multiple questions to provide to the patient in a subsequent user interaction, as the interaction workflow controls presentation of the multiple questions to the patient, and as a number, type, or order of the multiple questions is customized to the patient based on the attributes. In a first example, a priority is determined for each question of the multiple questions, as the number, type, or order of the multiple questions is further customized to the patient based on the priority of each question. In a second example (combinable with the first example), a complexity is determined for each question of the multiple questions, as the number, type, or order of the multiple questions is further customized to the patient based on the complexity of each question. In a third example (combinable with the first and second examples), a variability between the attributes of the user interactions and expected attributes of the user interactions may be determined, to then customize the task to the patient based on the determined variability.

The method 1100 continues at 1110 by controlling an interaction workflow to implement the task (e.g., in a software platform used by the patient, caregiver, or clinician). For instance, the task may include a scheduled time period to be presented in the software platform, and this scheduled time period may include a start time and an end time. In further examples, the scheduled time period may be customized to the patient based on the identified attributes.

In further examples, the method 1100 continues at 1112 by optionally implementing or modifying a programming therapy with the neurostimulation device, based on a patient state determined from the user interaction data and/or the interaction workflow. In an example, additional operations may be used to identify a patient state based on one or more of the user feedback or objective data associated with the patient, and then to associate the patient state with at least one neurostimulation program used for the neurostimulation treatment. As an example, such objective data may include data from a wearable such as accelerometry, steps, activity, sleep, mood, stress, heart rate, intensity minutes, effective mobility, etc. Also, once the patient state and/or the at least one neurostimulation program is determined, the interaction workflow itself (or a future interaction workflow) can be modified based on the patient state or the at least one neurostimulation program.

In an example, implementing or modifying a programming therapy includes controlling a closed-loop programming therapy of the neurostimulation device based on the identified patient state, such as based on determined characteristics the patient state that relate to one or more of: sleep, actigraphy (e.g., objectively measuring sleep parameters and cycles of activity and rest), accelerometry (e.g., physical activity levels based on accelerometers or like sensors), pain, movement, stress, disease-related symptoms (e.g., from movement disorders), emotional state, medication state, or specific activity, during or in connection with use of the at least one neurostimulation program. As a specific example, the closed-loop programming therapy may cause an automatic change to neurostimulation programming settings on the neurostimulation device, and as the automatic change to the neurostimulation programming settings controls one or more of: pulse patterns, pulse shapes, a spatial location of pulses, electric fields or activating functions of active electrodes, waveform shapes, or a spatial location of waveform shapes, for modulated energy provided with a plurality of leads of the neurostimulation device.

FIG. 12 illustrates, by way of example, a block diagram of an embodiment of a system 1200 (e.g., a computing system) for performing analysis of user interaction data (e.g., answers to patient questionnaires and other inputs of a user interface 1210) in connection with the data processing operations discussed above. The system 1200 may be integrated with or coupled to a computing device, a remote control device, patient programmer device, clinician programmer device, program modeling system, or other external device, deployed with neurostimulation treatment. In some examples, the system 1200 may be a networked device (server) connected via a network (or combination of networks) which communicates to one or more devices (clients) using a communication interface 1208 (e.g., communication hardware which implements software network interfaces and services). The network may include local, short-range, or long-range networks, such as Bluetooth, cellular, IEEE 802.11 (Wi-Fi), or other wired or wireless networks.

The system 1200 includes a processor 1202 and a memory 1204, which can be optionally included as part of user input/output data processing circuitry 1206. The processor 1202 may be any single processor or group of processors that act cooperatively. The memory 1204 may be any type of memory, including volatile or non-volatile memory. The memory 1204 may include instructions, which when executed by the processor 1202, cause the processor 1202 to implement data processing, or to enable other features of the user input/output data processing circuitry 1206. Thus, electronic operations in the system 1200 may be performed by the processor 1202 or the circuitry 1206.

For example, the processor 1202 or circuitry 1206 may implement any of the user-based features of the method 1100 to obtain and process user interaction activity, to generate user interface displays, and to control further user interaction workflows. It will be understood that the processor 1202 or circuitry 1206 may also implement aspects of the logic and processing described above, for use in various forms of closed-loop and partially-closed-loop device programming or related device actions.

FIG. 13 illustrates, by way of example, a block diagram of an embodiment of a system 1300 (e.g., a computing system) implementing neurostimulation programming circuitry 1306 to cause programming of an implantable electrical neurostimulation device, for accomplishing the therapy objectives in a human subject based on neurostimulation programming as discussed herein. The system 1300 may be operated by a clinician, a patient, a caregiver, a medical facility, a research institution, a medical device manufacturer or distributor, and embodied in a number of different computing platforms. The system 1300 may be a remote control device, patient programmer device, program modeling system, or other external device, including a regulated device used to directly implement programming commands and modification with a neurostimulation device. In some examples, the system 1300 may be a networked device connected via a network (or combination of networks) to a computing system operating a user interface computing system using a communication interface 1308. The network may include local, short-range, or long-range networks, such as Bluetooth, cellular, IEEE 802.11 (Wi-Fi), or other wired or wireless networks.

The system 1300 includes a processor 1302 and a memory 1304, which can be optionally included as part of neurostimulation programming circuitry 1306. The processor 1302 may be any single processor or group of processors that act cooperatively. The memory 1304 may be any type of memory, including volatile or non-volatile memory. The memory 1304 may include instructions, which when executed by the processor 1302, cause the processor 1302 to implement the features of the neurostimulation programming circuitry 1306. Thus, the electronic operations in the system 1300 may be performed by the processor 1302 or the circuitry 1306.

The processor 1302 or circuitry 1306 may directly or indirectly implement neurostimulation operations associated with the method 1100, including the use of neurostimulation device programming based on user interaction workflows (operation 1112).

The processor 1302 or circuitry 1306 may further provide data and commands to assist the processing and implementation of the programming using communication interface 1308 or a neurostimulation device interface 1310. It will be understood that the processor 1302 or circuitry 1306 may also implement other aspects of the device data processing or device programming functionality described above.

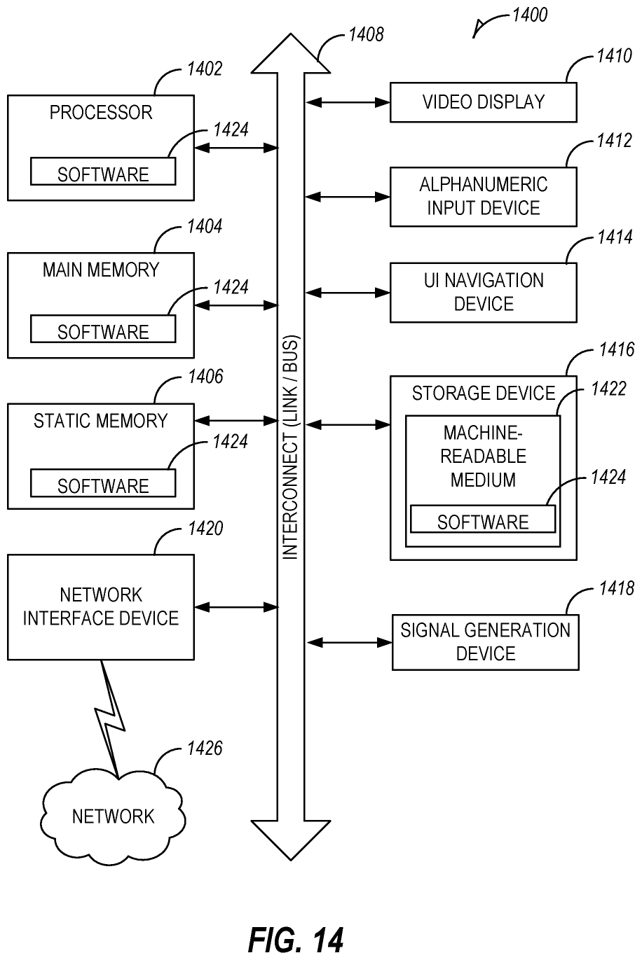

FIG. 14 is a block diagram illustrating a machine in the example form of a computer system 1400, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1400 includes at least one processor 1402 (e.g., a central processing unit (CPU), a graphics processing unit (GPU) or both, processor cores, compute nodes, etc.), a main memory 1404 and a static memory 1406, which communicate with each other via a link 1408 (e.g., bus). The computer system 1400 may further include a video display unit 1410, an alphanumeric input device 1412 (e.g., a keyboard), and a user interface (UI) navigation device 1414 (e.g., a mouse). In one embodiment, the video display unit 1410, input device 1412 and UI navigation device 1414 are incorporated into a touch screen display. The computer system 1400 may additionally include a storage device 1416 (e.g., a drive unit), a signal generation device 1418 (e.g., a speaker), a network interface device 1420, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 14 (such as a GPU, video display unit, keyboard, etc.).

The storage device 1416 includes a machine-readable medium 1422 on which is stored one or more sets of data structures and instructions 1424 (e.g., software) embodying or utilized by any one or more of the methodologies or functions described herein. The instructions 1424 may also reside, completely or at least partially, within the main memory 1404, static memory 1406, and/or within the processor 1402 during execution thereof by the computer system 1400, with the main memory 1404, static memory 1406, and the processor 1402 also constituting machine-readable media.

While the machine-readable medium 1422 is illustrated in an example embodiment to be a single medium, the term “machine-readable medium” may include a single medium or multiple media (e.g., a centralized or distributed database, and/or associated caches and servers) that store the one or more instructions 1424. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1424 may further be transmitted or received over a communications network 1426 using a transmission medium via the network interface device 1420 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A computing device to analyze user interaction associated with a neurostimulation treatment, the device comprising:
one or more processors; and
one or more memory devices comprising instructions, which when executed by the one or more processors, cause the one or more processors to:
receive user interaction data associated with a patient undergoing the neurostimulation treatment, wherein the user interaction data indicates attributes of one or more user interactions in a software platform that collect user feedback relating to the neurostimulation treatment;
identify attributes of the one or more user interactions, from the user interaction data;
generate a task to collect additional user input relating to the neurostimulation treatment, the task being customized to the patient based on the attributes of the one or more user interactions; and
control an interaction workflow to perform the task in the software platform.

2. The computing device of claim 1, wherein the user feedback is collected from the patient using one or more questions during each session of the one or more user interactions, and wherein one or more answers corresponding to the one or more questions provide a state of the patient undergoing the neurostimulation treatment.

3. The computing device of claim 2, wherein the attributes of the one or more user interactions relate to at least one of:
a number of tasks completed in each session;
a number of questions completed in each session;
an ongoing or historical usage of the software platform;
an ongoing or historical pattern of use of the software platform;
an amount of time of engagement in each session;
an amount of time of engagement with respective types of questions in each session;
a time of day of engagement in each session; or
a pattern of engagement in each session.

4. The computing device of claim 1, wherein the task includes multiple questions to provide to the patient in a subsequent user interaction, wherein the interaction workflow controls presentation of the multiple questions to the patient, and wherein a number, type, or order of the multiple questions is customized to the patient based on the attributes.

5. The computing device of claim 4, wherein a priority is determined for each question of the multiple questions, and wherein the number, type, or order of the multiple questions is further customized to the patient based on the priority of each question.

6. The computing device of claim 4, wherein a complexity is determined for each question of the multiple questions, and wherein the number, type, or order of the multiple questions is further customized to the patient based on the complexity of each question.

7. The computing device of claim 1, wherein the task includes a scheduled time period to be presented in the software platform, wherein the scheduled time period includes a start time and an end time, and wherein the scheduled time period is customized to the patient based on the attributes.

8. The computing device of claim 1, wherein the interaction workflow is a patient interaction workflow to occur with the patient, and wherein the software platform is implemented on a patient computing device provided by a personal computer, tablet, smartphone, remote control, or wearable device.

9. The computing device of claim 1, wherein the instructions further cause the one or more processors to:
identify a patient state based on one or more of the user feedback or objective data associated with the patient;
cause a change to the interaction workflow based on the identified patient state; and
cause a change in a closed-loop programming therapy of a neurostimulation device based on the identified patient state, wherein the patient state relates to one or more of: sleep, actigraphy, accelerometry, pain, movement, stress, disease-related symptoms, emotional state, medication state, or activity during use of at least one neurostimulation program.

10. The computing device of claim 9, wherein the closed-loop programming therapy causes an automatic change to neurostimulation programming settings on the neurostimulation device, and
wherein the automatic change to the neurostimulation programming settings controls one or more of: pulse patterns, pulse shapes, a spatial location of pulses, electric fields or activating functions of active electrodes, waveform shapes, or a spatial location of waveform shapes, for modulated energy provided with a plurality of leads of the neurostimulation device.

11. A method for analyzing user interaction associated with a neurostimulation treatment, comprising:
receiving user interaction data associated with a patient undergoing the neurostimulation treatment, wherein the user interaction data indicates attributes of one or more user interactions in a software platform that collect user feedback relating to the neurostimulation treatment;
identifying attributes of the one or more user interactions, from the user interaction data;
generating a task to collect additional user input relating to the neurostimulation treatment, the task being customized to the patient based on the attributes of the one or more user interactions; and
controlling an interaction workflow to perform the task in the software platform.

12. The method of claim 11, wherein the user feedback is collected from the patient using one or more questions during each session of the one or more user interactions, and wherein one or more answers corresponding to the one or more questions provide a state of the patient undergoing the neurostimulation treatment.

13. The method of claim 12, wherein the attributes of the one or more user interactions relate to at least one of:
a number of tasks completed in each session;
a number of questions completed in each session;
an ongoing or historical usage of the software platform;
an ongoing or historical pattern of use of the software platform;
an amount of time of engagement in each session;
an amount of time of engagement with respective types of questions in each session;
a time of day of engagement in each session; or
a pattern of engagement in each session.

14. The method of claim 11, wherein the task includes multiple questions to provide to the patient in a subsequent user interaction, wherein the interaction workflow controls presentation of the multiple questions to the patient, and wherein a number, type, or order of the multiple questions is customized to the patient based on the attributes.

15. The method of claim 14, wherein a priority is determined for each question of the multiple questions, and wherein the number, type, or order of the multiple questions is further customized to the patient based on the priority of each question.

16. The method of claim 14, wherein a complexity is determined for each question of the multiple questions, and wherein the number, type, or order of the multiple questions is further customized to the patient based on the complexity of each question.

17. The method of claim 11, wherein the task includes a scheduled time period to be presented in the software platform, wherein the scheduled time period includes a start time and an end time, and wherein the scheduled time period is customized to the patient based on the attributes.

18. The method of claim 11, wherein the interaction workflow is a patient interaction workflow to occur with the patient, and wherein the software platform is implemented on a patient computing device provided by a personal computer, tablet, smartphone, remote control, or wearable device.

19. The method of claim 11, further comprising:
identifying a patient state based on one or more of the user feedback or objective data associated with the patient;
causing a change to the interaction workflow based on the identified patient state; and
causing a change in a closed-loop programming therapy of a neurostimulation device based on the identified patient state, wherein the patient state relates to one or more of: sleep, actigraphy, accelerometry, pain, movement, stress, disease-related symptoms, emotional state, medication state, or activity during use of at least one neurostimulation program.

20. The method of claim 19, wherein the closed-loop programming therapy causes an automatic change to neurostimulation programming settings on the neurostimulation device, and
wherein the automatic change to the neurostimulation programming settings controls one or more of: pulse patterns, pulse shapes, a spatial location of pulses, electric fields or activating functions of active electrodes, waveform shapes, or a spatial location of waveform shapes, for modulated energy provided with a plurality of leads of the neurostimulation device.
